(19) United States
(12) Patent Application Publication
(10) Pub. No.: US 2011/0186831 A1
(43) Pub. Date: Aug. 4, 2011

(54) AROMATIC AMINE DERIVATIVE AND ORGANIC ELECTROLUMINESCENCE ELEMENT USING SAME

(75) Inventor: Yumiko Mizuki, Chiba (JP)

(73) Assignee: IDEMITSU KOSAN CO., LTD., Tokyo (JP)

(21) Appl. No.: 13/057,987

(22) PCT Filed: Aug. 12, 2009
(86) PCT No.: PCT/JP2009/064247

§ 371 Date: Apr. 14, 2011

(30) Foreign Application Priority Data
Aug. 12, 2008 (JP) 2008-208052

(51) Int. Cl. H01L 51/54; C07C 211/57; C07F 7/10; C07D 215/38

(52) U.S. Cl. 257/40; 564/427; 556/413; 546/171; 257/E51.024

(57) ABSTRACT

An aromatic amine derivative represented by the following formula (1):

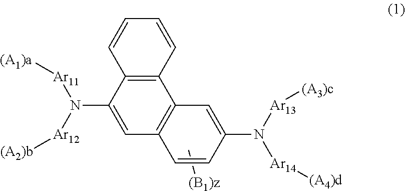

### TECHNICAL FIELD

The invention relates to an aromatic amine derivative and an organic electroluminescence device using the same. In particular, the invention relates to an organic electroluminescence device which emits blue light with a high chromatic purity at a high luminous efficiency, and an aromatic amine derivative realizing it.

### BACKGROUND ART

An organic electroluminescence device (hereinafter referred to as an “organic EL device”) utilizing an organic substance has a promising feature as an inexpensive, large-area full-color solid light-emitting display element, and many developments have been made on this type of organic EL device. Normally, an EL device is formed of an emitting layer and a pair of opposing electrodes disposing the emitting layer. Emission is a phenomenon that, when an electric field is applied between the both electrodes, electrons are injected from the cathode and holes are injected from the anode, these electrons are then recombined with the holes in the emitting layer, thereby to cause an excited state, and energy is discharged as light when the excited state is returned to the ground state.

Conventional organic EL devices have a higher driving voltage than an inorganic light-emitting diode. The luminance and luminous efficiency thereof are also low, and their properties tend to lower significantly. For these reasons, conventional organic EL devices have not been put in a practical use. Although recent organic EL devices have been improved gradually, further improvement in luminous efficiency and prolongation in life time has been demanded.

For example, a technology is disclosed in which a single monoanthracene compound is used as an organic emitting material (Patent Document 1). However, in this technology, a luminance of only 1650 cd/m² is obtained at a current density of 165 mA/cm², for example, and the efficiency is significantly low, i.e., 1 cd/A. This technology is, hence, not practical.

Further, a technology is disclosed in which a single bisanthracene compound is used as an organic emitting material (Patent Document 2). However, even in this technology, the efficiency is as low as about 1 to 3 cd/A, and there is an increasing demand for an improvement for practical use.

On the other hand, an organic EL device is proposed which has a long life in which a distryl compound is used as an organic emitting material and strylamine or the like is added thereto (Patent Document 3). However, development of a device with a further high efficiency has been demanded.

Also disclosed is a technology in which a mono- or bisanthracene compound and a distryl compound are used as an organic light-emitting medium layer (Patent Document 4). However, development of a device with a higher chromatic purity has been demanded.

### RELATED ART DOCUMENTS

### Patent Documents

Patent Document 1: JP-A-H11-3782
Patent Document 2: JP-A-H08-12600
Patent Document 3: WO94/006157
Patent Document 4: JP-A-2001-284050

### SUMMARY OF THE INVENTION

The invention has been made in order to solve the above-mentioned subject, and an object thereof is to provide an organic EL device capable of obtaining blue emission with a high chromatic purity at a high luminous efficiency and an aromatic amine derivative for realizing it.

The inventors of the present application have found that an organic EL device in which an aromatic amine derivative having phenanthrene as a central skeleton is used emits blue light with a high chromatic purity and has a high luminous efficiency. The invention has been made based on this finding.

According to the invention, the following aromatic amine derivative or the like can be provided.

1. An aromatic amine derivative represented by the following formula (1):

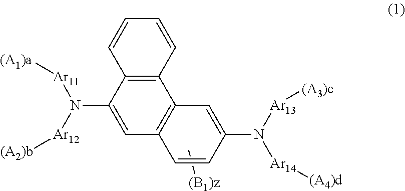

wherein Ar₁₁ to Ar₁₄ are independently a substituted or unsubstituted aryl group having 5 to 50 ring carbon atoms, or a substituted or unsubstituted heterocyclic group having 5 to 50 ring carbon atoms,

A₁ to A₄ are independently a hydrogen atom, a substituted or unsubstituted alkyl group having 1 to 50 carbon atoms, a substituted or unsubstituted aryl group having 5 to 50 ring carbon atoms, a substituted or unsubstituted aralkyl group having 6 to 50 ring carbon atoms, a substituted or unsubstituted cycloalkyl group having 3 to 50 ring carbon atoms, a substituted or unsubstituted alkoxyl group having 1 to 50 carbon atoms, a substituted or unsubstituted aryloxy group having 5 to 50 carbon atoms, a substituted or unsubstituted arylamino group having 5 to 50 ring carbon atoms, a substituted or unsubstituted alkylamino group having 1 to 20 carbon atoms, a substituted or unsubstituted heterocyclic group having 5 to 50 ring carbon atoms, a substituted or unsubstituted silyl group, a cyano group or a halogen atom,

B₁s are a hydrogen atom, a substituted or unsubstituted alkyl group having 1 to 50 carbon atoms, a substituted or unsubstituted aryl group having 5 to 50 ring carbon atoms, a substituted or unsubstituted aralkyl group having 6 to 50 ring carbon atoms or a substituted or unsubstituted cycloalkyl group having 3 to 50 ring carbon atoms,

a to d are independently an integer of 0 to 5 and when each of a to d is 2 or more, A₁s to A₄s may independently be the same or different and may bond to each other to form a saturated or unsaturated ring, and

z is an integer of 0 to 8 and when z is 2 or more, B₁s may independently be the same or different.

2. The aromatic amine derivative according to 1, wherein Ar₁₁ to Ar₁₄ in the formula (1) are independently a phenyl group or a naphthyl group.
3. The aromatic amine derivative according to 1 or 2, wherein A₁ to A₄ in the formula (1) are independently an alkyl group having 1 to 4 carbon atoms or an aryl group having 6 to 10 ring carbon atoms, and a to d are independently 1 or 2.
4. The aromatic amine derivative according to any of 1 to 3, wherein B₁s in the formula (1) are independently a hydrogen atom, an alkyl group having 1 to 4 carbon atoms or an aryl group having 6 to 10 ring carbon atoms, and z is 1 to 4.
5. The aromatic amine derivative according to any of 1 to 4, which is a doping material for an organic electroluminescence device.
6. An organic electroluminescence device comprising:

an anode, a cathode, and

one or plural organic thin film layers comprising at least an emitting layer between the anode and the cathode,

wherein at least one of the organic thin film layers comprises the aromatic amine derivative according to any of 1 to 5 singly or in the form of a mixture.

7. The organic electroluminescence device according to 6, wherein the emitting layer comprises the aromatic amine derivative according to any of 1 to 5 singly or in the form of a mixture.
8. The organic electroluminescence device according to 6, wherein the emitting layer comprises the aromatic amine derivative according to any of 1 to 5 in an amount of 0.1 to 20 weight %.
9. The organic electroluminescence device according to any of 6 to 8, which emits blue light.

By using the aromatic amine derivative of the invention, an organic EL device capable of obtaining blue emission with a high chromatic purity at a high luminous efficiency has been realized.

### BEST MODE FOR CARRYING OUT THE INVENTION

An aromatic amine derivative of the invention is a compound represented by the following formula (1):

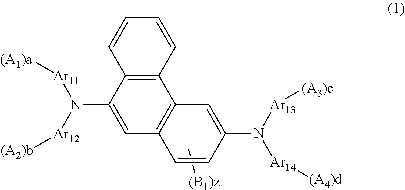

In the formula (1), Ar₁₁ to Ar₁₄ are independently a substituted or unsubstituted aryl group having 5 to 50 ring carbon atoms, or a substituted or unsubstituted heterocyclic group having 5 to 50 ring carbon atoms, and Ar₁₁ to Ar₁₄ are 1- to 3-substituted depending on substituents and A₁ to A₄.

The substituted or unsubstituted aryl group having 5 to 50 ring carbon atoms of Ar₁₁ to Ar₁₄ includes, though not limited thereto, phenyl, 1-naphthyl, 2-naphthyl, 1-anthryl, 2-anthryl, 9-anthryl, 9-(10-phenyl)anthryl, 9-(10-naphthyl-1-yl)anthryl, 9-(10-naphthyl-2-yl)anthryl, 1-phenanthryl, 2-phenanthryl, 3-phenanthryl, 4-phenanthryl, 9-phenanthryl, 1-naphthacenyl, 2-naphthacenyl, 9-naphthacenyl, 1-pyrenyl, 2-pyrenyl, 4-pyrenyl, 2-biphenylyl, 3-biphenylyl, 4-biphenylyl, p-terphenyl-4-yl, p-terphenyl-3-yl, p-terphenyl-2-yl, m-terphenyl-4-yl, m-terphenyl-3-yl, m-terphenyl-2-yl, o-tolyl, m-tolyl, p-tolyl, p-t-butylphenyl, 3-methyl-2-naphthyl, 4-methyl-1-naphthyl and 4-methyl-1-anthryl.

In the invention, the term “ring carbon atom” means a carbon atom forming a saturated ring, unsaturated ring or aromatic ring. The term “ring atom” means a carbon atom and hetero atom forming a hetero ring (including a saturated ring, unsaturated ring and aromatic ring).

In respect of stability, Ar₁₁ to Ar₁₄ are independently a substituted or unsubstituted aryl group having 6 to 16 ring carbon atoms. In particularly, phenyl, 1-naphthyl, 2-naphthyl, 9-(10-phenyl)anthryl, 9-(10-naphthyl-1-yl)anthryl, 9-(10-naphthyl-2-yl)anthryl, 9-phenanthryl, 1-pyrenyl, 2-pyrenyl, 4-pyrenyl, 2-biphenylyl, 3-biphenylyl, 4-biphenylyl, o-tolyl, m-tolyl, p-tolyl and p-t-butylphenyl are preferable.

The substituted or unsubstituted heterocyclic group having 5 to 50 ring carbon atoms of Ar₁₁ to Ar₁₄ includes, though not limited thereto, residues such as imidazole, benzimidazole, pyrrole, furan, thiophene, benzothiophene, oxadiazoline, indoline, carbazole, pyridine, quinoline, isoquinoline, benzoquinone, pyralozine, imidazolidine, piperidine, dibenzofuran, benzofuran and dibenzothiophene.

In the formula (1), A₁to A₄ are independently a hydrogen atom, a substituted or unsubstituted alkyl group having 1 to 50 (preferably 1 to 20, and particularly preferably 1 to 4) carbon atoms, a substituted or unsubstituted aryl group having 5 to 50 (preferably 5 to 20, and particularly preferably 6 to 10) ring carbon atoms, a substituted or unsubstituted aralkyl group having 6 to 50 (preferably 6 to 20) ring carbon atoms, a substituted or unsubstituted cycloalkyl group having 3 to 50 (preferably 5 to 12) ring carbon atoms, a substituted or unsubstituted alkoxyl group having 1 to 50 (preferably 1 to 6) carbon atoms, a substituted or unsubstituted aryloxy group having 5 to 50 (preferably 5 to 18) ring carbon atoms, a substituted or unsubstituted arylamino group having 5 to 50 (preferably 5 to 18) ring carbon atoms, a substituted or unsubstituted alkylamino group having 1 to 20 (preferably 1 to 6) carbon atoms, a substituted or unsubstituted heterocyclic group having 5 to 50 (preferably 5 to 20) ring carbon atoms, a substituted or unsubstituted silyl group, a cyano group or a halogen atom,

The substituted or unsubstituted alkyl group of A₁ to A₄ includes, though not limited thereto, methyl, ethyl, propyl, isopropyl, butyl, sec-butyl, tert-butyl, pentyl, hexyl, heptyl, octyl, stearyl, 2-phenylisopropyl, trichloromethyl, trifluoromethyl, benzyl, α-phenoxybenzyl, α,α-dimethylbenzyl, α,α-methylphenylbenzyl, α,α-ditrifluoromethylbenzyl, triphenylmethyl, and α-benzyloxybenzyl groups.

In respect of stability, among the above-mentioned alkyl groups, preferable is the alkyl group having 1 to 4 carbon atoms such as methyl, ethyl, propyl, isopropyl, butyl, sec-butyl and tert-butyl groups.

The substituted or unsubstituted aryl group of A₁ to A₄ includes, though not limited thereto, phenyl, 2-methylphenyl, 3-methylphenyl, 4-methylphenyl, 4-ethylphenyl, biphenyl, 4-methylbiphenyl, 4-ethylbiphenyl, 4-cyclohexylbiphenyl, terphenyl, 3,5-dichlorophenyl, 1-naphthyl, 2-naphthyl, 5-methylnaphthyl, anthryl, and pyrenyl groups.

In respect of stability, among the above-mentioned aryl groups, the aryl group having 6 to 10 ring carbon atoms is preferable.

The substituted or unsubstituted aralkyl group of A₁to A₄ includes, though not limited thereto, benzyl, 1-phenylethyl, 2-phenylethyl, 1-phenylisopropyl, 2-phenylisopropyl, phenyl-t-butyl, α-naphthylmethyl, 1-α-naphthylethyl, 2-α-naphthylethyl, 1-α-naphthylisopropyl, 2-α-naphthylisopropyl, β-naphthylmethyl, 1-β-naphthylethyl, 2-β-naphthylethyl, 1-β-naphthylisopropyl, 2-β-naphthylisopropyl, 1-pyrrolylmethyl, 2-(1-pyrrolyl)ethyl, p-methylbenzyl, m-methylbenzyl, o-methylbenzyl, p-chlorobenzyl, m-chlorobenzyl, o-chlorobenzyl, p-bromobenzyl, m-bromobenzyl, o-bromobenzyl, p-iodobenzyl, m-iodobenzyl, o-iodobenzyl, p-hydroxybenzyl, m-hydroxybenzyl, o-hydroxybenzyl, p-aminobenzyl, m-aminobenzyl, o-aminobenzyl, p-nitrobenzyl, m-nitrobenzyl, o-nitrobenzyl, p-cyanobenzyl, m-cyanobenzyl, o-cyanobenzyl, 1-hydroxy-2-phenylisopropyl, and 1-chloro-2-phenylisopropyl groups.

The cycloalkyl group of A₁ to A₄ includes, though not limited thereto, cyclopropyl, cyclobutyl, cyclopentyl, cyclohexyl, cycloheptyl, cyclooctyl, cyclononyl, bicycloheptyl, bicyclooctyl, tricycloheptyl, and adamantyl groups. Among these, cyclopentyl, cyclohexyl, cycloheptyl, bicycloheptyl, bicyclooctyl and adamantyl groups are preferable.

The alkoxyl group of A₁ to A₄ includes, though not limited thereto, methoxy, ethoxy, propoxy, isopropoxy, butoxy, isobutoxy, sec-butoxy, tert-butoxy, various pentyloxy, and various hexyloxy groups.

The substituted or unsubstituted aryloxy group of A₁ to A₄ includes, though not be limited thereto, phenoxy, tolyloxy, and naphthyloxy groups.

The substituted or unsubstituted arylamino group of A₁ to A₄ includes, though not be limited thereto, diphenylamino, ditolylamino, dinaphthylamino, and naphthylphenylamino groups.

The alkylamino group of A₁ to A₄ includes, though not limited thereto, dimethylamino, diethylamino, and dihexylamino groups.

The substituted or unsubstituted heterocyclic group of A₁ to A₄ includes, though not limited thereto, residues such as imidazole, benzimidazole, pyrrole, furan, thiophene, benzothiophene, oxadiazoline, indoline, carbazole, pyridine, quinoline, isoquinoline, benzoquinone, pyralozine, imidazolidine, piperidine, dibenzofuran, benzofuran and dibenzothiophene.

The substituent of the silyl group of A₁ to A₄ includes an alkyl group having 1 to 20 carbon atoms, an aryl group having 6 to 14 carbon atoms, and an alkoxy group having 1 to 20 carbon atoms. Examples of the alkyl group having 1 to 20 carbon atoms include methyl, ethyl, n-propyl, isopropyl, n-butyl, isobutyl, s-butyl, t-butyl, and pentyl groups, with an alkyl group having 1 to 5 carbon atoms being preferable. As the aryl group having 6 to 14 carbon atoms, a phenyl group, a tosyl group, a naphthyl group, and an anthryl group can be given, for example, with an aryl group having 6 to 10 carbon atoms being preferable. As the alkoxy group having 1 to 20 carbon atoms, a methoxy group, an ethoxy group, a propoxy group and a butoxy group can be given, for example, with an alkoxyl group having 1 to 5 carbon atoms being preferable.

As the halogen atom of A₁ to A₄, a fluorine atom, a chlorine atom, a bromine atom or the like can be given.

In the formula (1), a to d are independently an integer of 0 to 5. An integer of 0 to 3 is preferable, with an integer of 0 to 2 being particularly preferable. In respect of stability, it is particularly preferred that a to d be independently 1 or 2.

When each of a to d is 2 or more, A₁s to A₄s are independently the same or different, and may be combined with each other to form a saturated or unsaturated ring.

Examples of such ring include a cycloalkane having 4 to 12 carbon atoms such as cyclobutane, cyclopentane and cyclohexane, a cycloalkene having 4 to 12 carbon atoms such as cyclobutene, cyclopentene, cyclohexene, cycloheptene and cyclooctene, and a cycloalkadiene having 6 to 12 carbon atoms such as cyclohexadiene, cycloheptadiene and cyclooctadiene.

As the substituent of A₁ to A₄ mentioned above, an aryl group having 5 to 50 ring carbon atoms, an alkyl group having 1 to 50 carbon atoms, an alkoxy group having 1 to 50 carbon atoms, an aralkyl group having 6 to 50 ring carbon atoms, an aryloxy group having 5 to 50 ring carbon atoms, an arylthio group having 5 to 50 ring carbon atoms, an alkoxycarbonyl group having 1 to 50 carbon atoms, an amino group, a halogen atom, a cyano group, a nitro group, a hydroxyl group, a carboxyl group or the like can be given. Specific examples of each group are the same as those exemplified above as the examples of A₁ to A₄.

In the formula (1), at least one of a to d is an integer of 1 or more. In such a case, at least one of A₁ to A₄ is a substituted or unsubstituted cycloalkyl group having 3 to 50 ring carbon atoms. It is preferred that this cycloalkyl group be a cyclopentyl group, a cyclohexyl group, a cycloheptyl group, a bicycloheptyl group, a bicylooctyl group or an adamantyl group.

B₁ is a hydrogen atom, a substituted or unsubstituted alkyl group having 1 to 50 carbon atoms, a substituted or unsubstituted aryl group having 5 to 50 ring carbon atoms, a substituted or unsubstituted aralkyl group having 6 to 50 ring carbon atoms or a substituted or unsubstituted cycloalkyl group having 3 to 50 ring carbon atoms.

Specific examples of each group are the same as those exemplified above as the examples of A₁ to A₄.

In respect of stability, of these group, it is preferred that B₁s be independently a hydrogen atom, an alkyl group having 1 to 4 carbon atoms or an aryl group having 6 to 10 ring carbon atoms.

z is an integer of 0 to 8, and if z is 2 or more, zs may independently be the same or different. In respect of stability, in particular, it is preferred that zs be independently an integer of 1 to 4, with an integer of 1 to 2 being particularly preferable.

Specific examples of the aromatic amine derivative of the invention represented by the formula (1) are given below, though the aromatic amine derivative of the invention is not limited to these exemplified compounds.

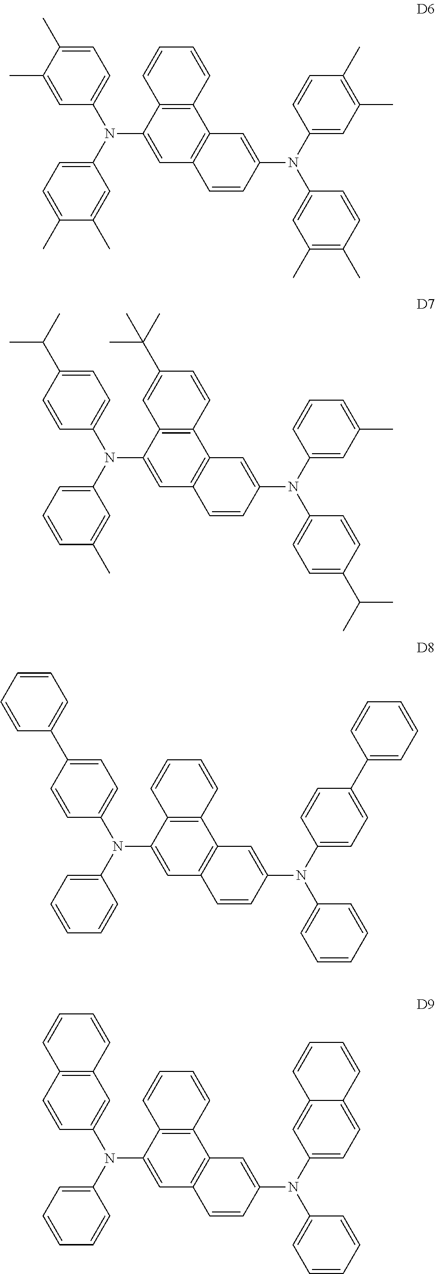

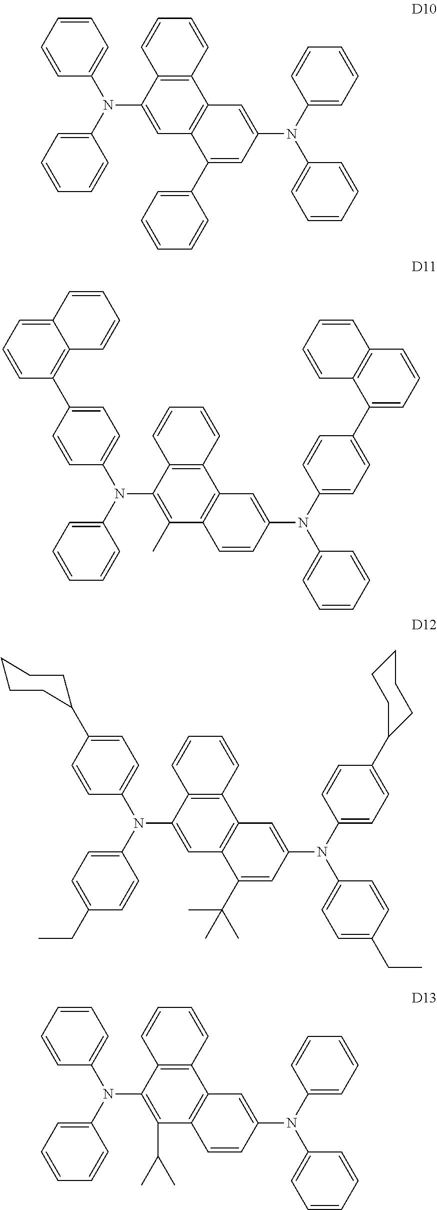

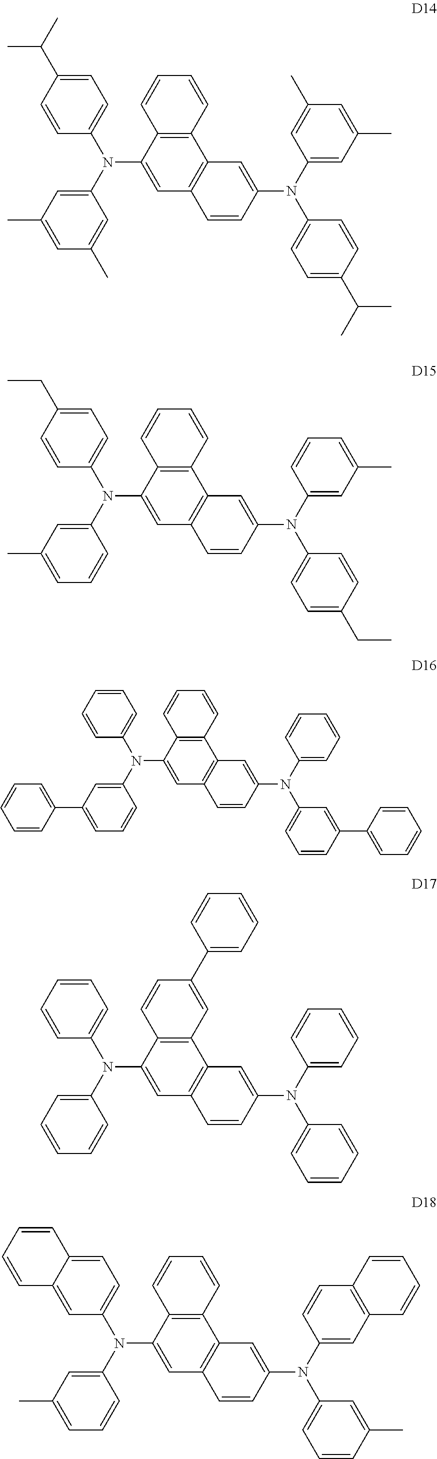

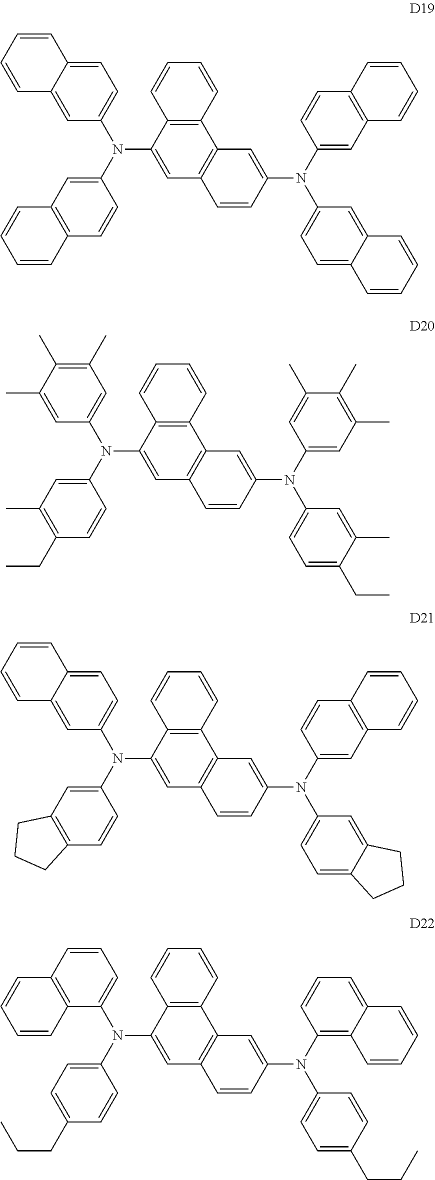

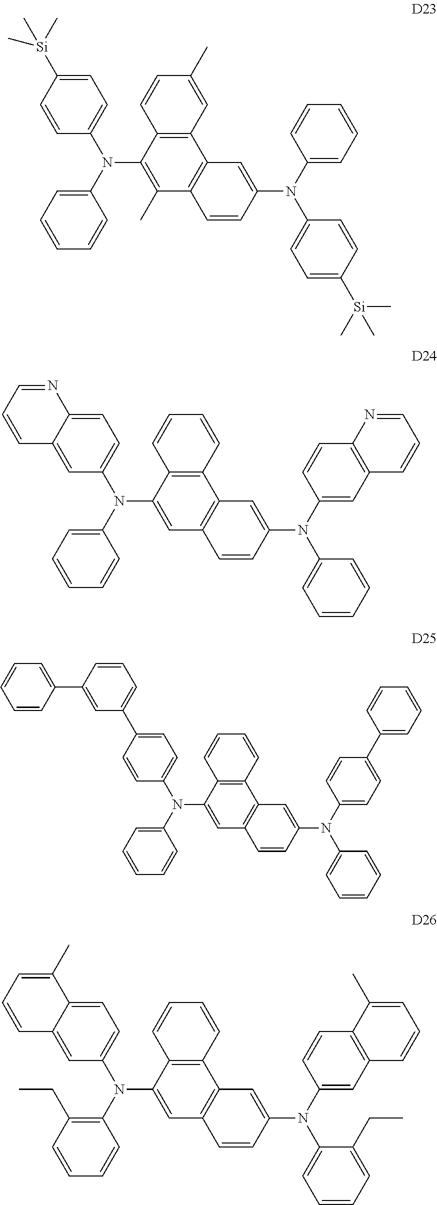

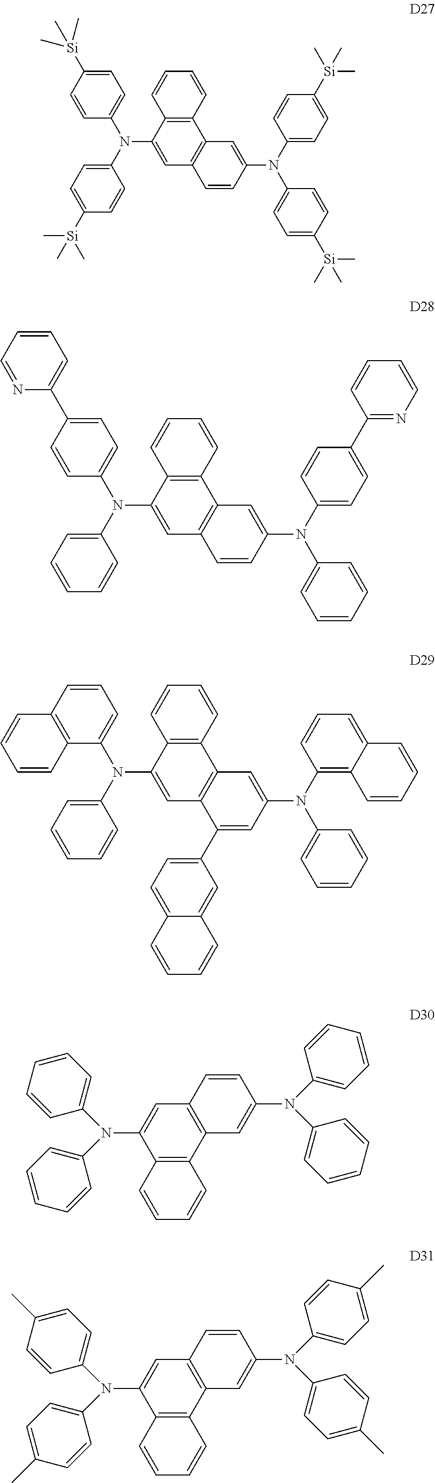

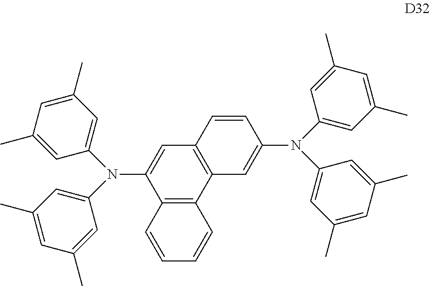

Regarding the aromatic amine derivative of the invention, 3,9-dibromophenanthrene as a mother skeleton can be produced by a known method described, for example, in J. Org. Chem., 11, 307 (1997) or the like. Subsequent to the production thereof, by a C—N coupling reaction (Buchwald-Hartwig reaction, or the like), the compound of the invention can be derived therefrom.

The aromatic amine derivative of the invention represented by the formula (1) has excellent hole-injecting and hole-transporting properties from a metal electrode or an organic thin film layer and excellent electron-injecting and electron-transporting properties from a metal electrode or an organic thin film layer, and hence, it is effectively used as an emitting material, in particular, a doping material, of the organic EL device. Other hole-transporting materials, electron-transporting materials or doping materials may be used.

The organic EL device of the invention is a device which comprises an anode, a cathode and one or plural organic thin film layers between the anode and the cathode. In the case of an organic EL device having a single organic thin film layer, an emitting layer is provided between the anode and the cathode. The emitting layer contains an emitting material. In addition to the emitting material, in order to allow holes injected from the anode or electrons injected from the cathode to be transported to the emitting material, a hole-injecting material or an electron-injecting material may be contained. Since the aromatic amine derivative represented by the formula (1) has a high emission properties, and excellent hole-injecting and hole-transporting properties and electron-injecting and electron-transporting properties, it can be used in an emitting layer as an emitting material or a doping material.

In the organic EL device of the invention, it is preferred that the emitting layer contain the aromatic amine derivative of the invention, and the content thereof is normally 0.1 to 20 wt %. It is further preferred that the content be 1 to 10 wt % in respect of chromaticity adjustment and stability. Since the aromatic amine derivative of the invention has a significantly high fluorescence quantum efficiency, excellent hole-transporting capability and electron-transporting capability in combination, and can form a uniform thin film, it is possible to form an emitting layer only of this aromatic amine derivative.

Further, as for the organic EL device of the invention, in the organic EL device in which two or more organic thin film layers including at least an emitting layer are disposed between the cathode and the anode, it is preferred that an organic layer composed mainly of the aromatic amine derivative of the invention be disposed between the anode and the emitting layer. As for this organic layer, a hole-injecting layer, a hole-transporting layer or the like can be given.

If the aromatic amine derivative of the invention is contained as a doping material, in respect of durability, it is preferred that a compound represented by the formulas (2a) and (2b) be contained as a host material. Hereinbelow, an explanation is made on the formulas (2a) and (2b).

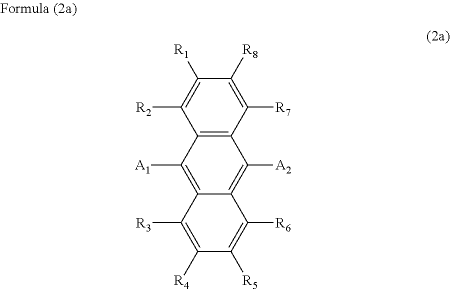

In the formula (2a), A₁ and A₂ are independently a group derived from a substituted or unsubstituted aromatic ring having 6 to 20 ring carbon atoms. The aromatic ring may be substituted by one or two or more substituents. The substituent is selected from a substituted or unsubstituted aryl group having 6 to 50 ring carbon atoms, a substituted or unsubstituted alkyl group having 1 to 50 carbon atoms, a substituted or unsubstituted cycloalkyl group having 3 to 50 carbon atoms, a substituted or unsubstituted alkoxy group having 1 to 50 carbon atoms, a substituted or unsubstituted aralkyl group having 6 to 50 carbon atoms, a substituted or unsubstituted aryloxy group having 5 to 50 ring atoms, a substituted or unsubstituted arylthio group having 5 to 50 ring atoms, a substituted or unsubstituted alkoxycarbonyl group having 1 to 50 carbon atoms, a substituted or unsubstituted silyl group, a carboxyl group, a halogen atom, a cyano group, a nitro group and a hydroxyl group. If the aromatic ring is substituted by two or more substituents, the substituents may be the same or different, and adjacent substituents may be bonded to each other to form a saturated or unsaturated ring structure.

R₁ to R₈ are independently selected from a hydrogen atom, a substituted or unsubstituted aryl group having 6 to 50 ring carbon atoms, a substituted or unsubstituted heteroaryl group having 5 to 50 ring atoms, a substituted or unsubstituted alkyl group having 1 to 50 carbon atoms, a substituted or unsubstituted cycloalkyl group having 3 to 50 carbon atoms, a substituted or unsubstituted alkoxy group having 1 to 50 carbon atoms, a substituted or unsubstituted aralkyl group having 6 to 50 carbon atoms, a substituted or unsubstituted aryloxy group having 5 to 50 ring atoms, a substituted or unsubstituted arylthio group having 5 to 50 ring atoms, a substituted or unsubstituted alkoxycarbonyl group having 1 to 50 carbon atoms, a substituted or unsubstituted silyl group, a carboxyl group, a halogen atom, a cyano group, a nitro group and a hydroxyl group.

In the formula (2a), it is preferred that A₁ and A₂ mentioned above be different groups.

In the above-mentioned formula (2a), at least one of A₁ and A₂ be a substituent having a substituted or unsubstituted fused ring group having 10 to 30 ring atoms.

It is preferred that the substituted or unsubstituted fused ring group having 10 to 30 ring atoms be a substituted or unsubstituted naphthalene ring.

The substituted or unsubstituted aryloxy group having 5 to 50 ring atoms and the substituted or unsubstituted arylthio group having 5 to 50 ring atoms of R₁ to R₈ and the substituent of the aromatic ring in the formula (2a) are respectively represented by —OY′ and —SY″. As examples of Y′ and Y″, the same groups as the substituted or unsubstituted aryl group having 6 to 50 ring atoms of R₁ to R₈ and the substituent of the aromatic ring can be given.

The substituted or unsubstituted alkoxycarbonyl group having 1 to 50 carbon atoms represented by R₁ to R₈ and the substituent of the aromatic ring in the formula (2a) are respectively represented by —COOZ. As examples of Z, the same groups as the substituted or unsubstituted alkyl group having 1 to 50 carbon atoms of R₁ to R₈ and the substituent of the aromatic ring can be given.

As the substituted or unsubstituted silyl group of R₁ to R₈ or the substituent of the aromatic ring in the formula (2a), a trimethylsilyl group, a triethylsilyl group, a t-butyldimethylsilyl group, a vinyldimethylsilyl group, a propyldimethylsilyl group, a triphenylsilyl group or the like can be given.

As the halogen atom of R₁ to R₈ and the substituent of the aromatic ring in the formula (2a), fluorine or the like can be given.

As the substituent for the group of R₁ to R₈ or the substituent of the aromatic ring in the formula (2a), a halogen atom, a hydroxyl group, a nitro group, a cyano group, an alkyl group, an aryl group, a cycloalkyl group, an alkoxy group, an aromatic heterocyclic group, an aralkyl group, an aryl group, a cycloalkyl group, an alkoxy group, an aromatic heterocyclic group, an aralkyl group, an aryloxy group, an arylthio group, an alkoxycarbonyl group, a carboxyl group or the like can be given.

It is preferred that the anthracene derivative represented by the formula (2a) be a compound having a structure represented by the following formula (2a′):

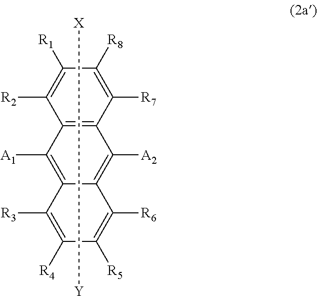

A₁ and A₂ and R₁ to R₈ in the formula (2a′) are independently the same as those in formula (2a), and the same specific examples can be given.

However, in the formula (2a′), groups do not symmetrically bond to 9 and 10 positions of the central anthracene with respect to the X-Y axis.

As the specific examples of the anthracene derivative represented by the formula (2a) used in the organic EL device of the invention, known various anthracene derivatives such as one having two anthracene skeletons in a molecule shown in paragraphs [0043] to [0063] of JP-A-2004-356033 or a compound having one anthracene skeleton shown on pages 27 to 28 of WO2005/061656 can be given.

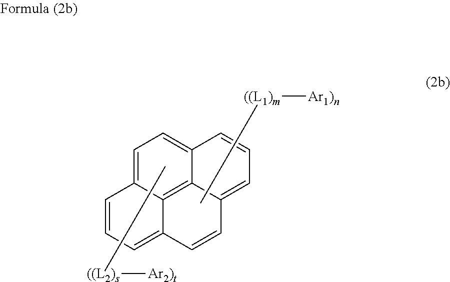

In the formula (2b), Ar₁ and Ar₂ are independently a substituted or unsubstituted aryl group having 6 to 50 ring carbon atoms;

L₁ and L₂ are independently a group selected from a substituted or unsubstituted phenylene group, a substituted or unsubstituted naphthalene group, a substituted or unsubstituted fluorenylene group and a substituted or unsubstituted dibenzosilolylene group;

m is an integer of 0 to 2, n is an integer of 1 to 4, s is an integer of 0 to 2 and t is an integer of 0 to 4; and

L₁ or Ar₂ bonds to any one position of 1 to 5 of pyrene, and L₂ or Ar₂ bonds to any one position of 6 to 10 of pyrene.

L₁ and L₂ in the formula (2b) are preferably selected from a substituted or unsubstituted phenylene group and a substituted or unsubstituted fluorenylene group.

As this substituent, the same groups as those exemplified for the above-mentioned aromatic group can be given.

In the invention, as the organic EL device in which the organic thin film layer is composed of plural layers, one in which an anode, a hole-injecting layer, an emitting layer and a cathode are sequentially stacked (anode/hole-injecting layer/emitting layer/cathode), one in which an anode, an emitting layer, an electron-injecting layer and a cathode are sequentially stacked (anode/emitting layer/electron-injecting layer/cathode), one in which an anode, a hole-injecting layer, an emitting layer, electron-injecting layer and a cathode are sequentially stacked (anode/hole-injecting layer/emitting layer/electron-injecting layer/cathode) or the like can be given.

If necessary, to the above-mentioned plural layers, in addition to the aromatic amine derivative of the invention, a further known emitting material, a doping material, a hole-injecting material or an electron-injecting material can be used. By allowing the organic thin film layer to be composed of plural layers, the organic EL device can be prevented from a lowering of luminance or lifetime due to quenching. If necessary, an emitting material, a doping material, a hole-injecting material or an electron-injecting material can be used in combination. Further, due to the use of a doping material, luminance or luminous efficiency can be improved or red or blue emission can be obtained. The hole-injecting layer, the emitting layer and the electron-injecting layer may respectively be formed of two or more layers. In such case, in the hole-injecting layer, a layer which injects holes from an electrode is referred to as a hole-injecting layer, and a layer which receives holes from the hole-injecting layer and transports the holes to the emitting layer is referred to as a hole-transporting layer. Similarly, in the electron-injecting layer, a layer which injects electrons from an electrode is referred to as an electron-injection layer and a layer which receives electrons from an electron-injecting layer and transports the electrons to the emitting layer is referred to as an electron-transporting layer. Each of these layers is selected and used according to each of the factors, i.e. the energy level, heat resistance, adhesiveness to the organic layer or the metal electrode or the like.

Examples of the host material or the doping material which can be used in the emitting layer together with the aromatic amine derivative of the invention include, though not limited thereto, fused multimeric aromatic compounds such as naphthalene, phenanthrene, rubrene, anthracene, tetracene, pyrene, perylene, chrysene, decacyclene, coronene, tetraphenylcyclopentadiene, pentaphenylcyclopentadiene, fluorene, spirofluorene, 9,10-diphenylanthracene, 9,10-bis(phenylethynyl)anthracene, 1,4-bis(9′-ethynylanthracenyl)benzene, and the derivatives thereof, organic metal complexes such as tris(8-quinolinolate)aluminum, bis-(2-methyl-8-quinolinolate)-4-(phenylphenolinate)aluminum, triarylamine derivatives, styrylamine derivatives, stilbene derivatives, coumarin derivatives, pyrane derivatives, oxazoline derivatives, benzothiazole derivatives, benzoxazole derivatives, benzimidazole derivatives, pyrazine derivatives, cinnamate derivatives, diketo-pyrrolo-pyrrole derivatives, acrylidone derivatives and quinacrylidone derivatives or the like.

As the hole-injecting material, a compound which can transport holes, exhibits hole-injecting effects from the anode and excellent hole-injection effect for the emitting layer or the emitting material, prevents excitons generated in the emitting layer from moving to the electron-injecting layer or the electron-injecting material, and has an excellent capability of forming a thin film is preferable. Specific examples thereof include, though not limited thereto, phthalocyanine derivatives, naphthalocyanine derivatives, porphyline derivatives, oxazole, oxadiazole, triazole, imidazole, imidazolone, imidazolethione, pyrazoline, pyrazolone, tetrahydroimidazole, oxazole, oxadiazole, hydrazone, acylhydrazone, polyarylalkane, stilbene, butadiene, benzidine-type triphenylamine, strylamine-type triphenylamine, diamine-type triphenylamine, derivatives thereof, and polymer materials such as polyvinyl carbazole, polysilane and conductive polymers.

Of the hole-injecting materials usable in the organic EL device of the invention, further effective hole-injecting materials are aromatic tertiary amine derivatives and phthalocyanine derivatives.

Examples of the aromatic tertiary amine derivative include, though not limited thereto, triphenylamine, tritolylamine, tolyldiphenylamine, N,N′-diphenyl-N,N′-(3-methylphenyl)-1,1′-biphenyl-4-4′-diamine, N,N,N′,N′-(4-methylphenyl)-1,1′-phenyl-4,4′-diamine, N,N,N′,N′-(4-methylphenyl)-1,1′-biphenyl-4,4′-diamine, N,N′-diphenyl-N,N′-dinaphthyl-1,1′-biphenyl-4,4′-diamine, N,N′-(methylphenyl)-N,N′-(4-n-butylphenyl)-phenanthrene-9,10-diamine, N,N-bis(4-di-4-tolylaminophenyl)-4-phenyl-cyclohexane or the like, or an oligomer or a polymer having these aromatic tertiary amine skeleton.

Examples of the phthalocyanine (Pc) derivative include, though not limited thereto, phthalocyanine derivatives and naphthalocyanine derivatives such as H₂Pc, CuPc, CoPc, NiPc, ZnPc, PdPc, FePc, MnPc, ClAlPc, ClGaPc, ClInPc, ClSnPc, Cl₂SiPc, (HO)AlPc, (HO)GaPc, VOPc, TiOPc, MoOPc and GaPc-O—GaPc.

In the organic EL device of the invention, it is preferred that a layer containing these aromatic tertiary amine derivatives and/or phthalocyanine derivatives, for example, the above-mentioned hole-transporting layer and/or the hole-injecting layer, be formed between the emitting layer and the anode.

As the electron-injecting material, a compound which can transport electrons, exhibits electron-injecting effects from the cathode and excellent electron-injection effect for the emitting layer or the emitting material, prevents excitons generated in the emitting layer from moving to the hole-injecting layer, and has an excellent capability of forming a thin film is preferable. Specific examples thereof include, though not limited thereto, fluorenone, anthraquinodimethane, diphenoquinone, thiopyran dioxide, oxazole, oxadiazole, triazole, imidazole, perylene tetracarboxylic acid, fluorenylidene methane, anthraquinodimethane, anthrone and the derivatives thereof. In addition, it is also possible to sensitize the hole-injecting material by adding an electron-accepting substance and to sensitize the electron-injecting material by adding an electron-donating material.

In the organic EL device of the invention, a further effective electron-injecting material is a metal complex compound and a nitrogen-containing five-membered ring derivative.

Examples of the metal complex compound include, though not limited thereto, 8-hydroxyquinolinate lithium, bis(8-hydroxyquinolinate)zinc, bis(8-hydroxyquinolinate)copper, bis(8-hydroxyquinolinate)manganese, tris(8-hydroxyquinolinate)aluminum, tris(2-methyl-8-hydroxyquinolinate)aluminum, tris(8-hydroxyquinolinate)gallium, bis(10-hydroxybenzo[h]quinolinate)beryllium, bis(10-hydroxybenzo[h]quinolinate)zinc, bis(2-methyl-8-quinolinate)chlorogallium, bis(2-methyl-8-quinolinate)(o-crezolate)gallium, bis(2-methyl-8-quinolinate)(1-naphtholate)aluminum and bis(2-methyl-8-quinolinate)(2-naphtholate)gallium.

As the above-mentioned nitrogen-containing five-membered ring derivative, oxazole, thiazole, oxadiazole, thiadiazole and triazole derivatives are preferable. Specific examples thereof include, though not limited thereto, 2,5-bis(1-phenyl)-1,3,4-oxazole, dimethylPOPOP, 2,5-bis(1-phenyl)-1,3,4-thiazole, 2,5-bis(1-phenyl)-1,3,4-oxadiazole, 2-(4′-tert-butylphenyl)-5-(4″-biphenyl)1,3,4-oxadiazole, 2,5-bis(1-naphthyl)-1,3,4-oxadiazole, 1,4-bis[2-(5-phenyloxadiazolyl)]benzene, 1,4-bis[2-(5-phenyloxadiazolyl)-4-tert-butylbenzene], 2-(4′-tert-butylphenyl)-5-(4″-biphenyl)-1,3,4-thiaziazole, 2,5-bis(1-naphthyl)-1,3,4-thiaziazole, 1,4-bis[2-(5-phenylthiazolyl)]benzene, 2-(4′-tert-butylphenyl)-5-(4″-biphenyl)-1,3,4-triazole, 2,5-bis(1-naphthyl)-1,3,4-triazole and 1,4-bis[2-(5-phenyltriazolyl)]benzene.

In the organic EL device of the invention, the emitting layer may contain, in addition to the above-mentioned aromatic amine derivative represented by the formula (1), at least one of an emitting material, a doping material, a hole-injecting material, and an electronic-injecting material in the same layer. Moreover, for improving stability of the organic EL device obtained by the invention to temperature, humidity, atmosphere, etc. it is also possible to prepare a protective layer on the surface of the device, and it is also possible to protect the entire device by applying silicone oil, resin, etc.

As the conductive material used in the anode of the organic EL device of the invention, a conductive material having a work function of more than 4 eV is suitable. Carbon, aluminum, vanadium, iron, cobalt, nickel, tungsten, silver, gold, platinum, palladium or the like, alloys thereof, oxidized metals which are used in an ITO substrate and a NESA substrate such as tin oxide and indium oxide and organic conductive resins such as polythiophene and polypyrrole are used. As the conductive material used in the cathode, a conductive material having a work function of smaller than 4 eV is suitable. Magnesium, calcium, tin, lead, titanium, yttrium, lithium, ruthenium, manganese, aluminum, and lithium fluoride or the like, and alloys thereof are used, but not limited thereto. Representative examples of the alloys include, though not limited thereto, magnesium/silver alloys, magnesium/indium alloys and lithium/aluminum alloys. The amount ratio of the alloy is controlled by the temperature of the deposition source, atmosphere, vacuum degree or the like, and an appropriate ratio is selected. If necessary, the anode and the cathode each may be composed of two or more layers.

In the organic EL device of the invention, in order to allow it to emit light efficiently, it is preferred that at least one of the surfaces be fully transparent in the emission wavelength region of the device. In addition, it is preferred that the substrate also be transparent. The transparent electrode is set such that predetermined transparency can be ensured by a method such as deposition or sputtering by using the above-mentioned conductive materials. It is preferred that the electrode on the emitting surface have a light transmittance of 10% or more. Although no specific restrictions are imposed on the substrate as long as it has mechanical and thermal strength and transparency, a glass substrate and a transparent resin film can be given. Examples of the transparent resin film include polyethylene, an ethylene-vinyl acetate copolymer, an ethylene-vinyl alcohol copolymer, polypropylene, polystyrene, polymethylmethacrylate, polyvinyl chloride, polyvinyl alcohol, polyvinyl butyral, nylon, polyether ether ketone, polysulfone, polyether sulfone, a tetrafluoroethylene-perfluoroalkyl vinyl ether copolymer, polyvinyl fluoride, a tetrafluoroethylene-ethylene copolymer, a tetrafluoroethylene-hexafluoropropylene copolymer, polychlorotrifluoroethylene, polyvinylidene fluoride, polyester, polycarbonate, polyurethane, polyimide, polyether imide, polyimide and polypropylene.

Each layer of the organic EL device of the invention can be formed by a dry film-forming method such as vacuum vapor deposition, sputtering, plasma, ion plating or the like or a wet film-forming method such as spin coating, dipping, flow coating or the like. Although the film thickness is not particularly limited, it is required to adjust the film thickness to an appropriate value. If the film thickness is too large, a large voltage is required to be applied in order to obtain a certain optical output, which results in a poor efficiency. If the film thickness is too small, pinholes or the like are generated, and a sufficient luminance cannot be obtained even if an electrical field is applied. The suitable film thickness is normally 5 nm to 10 μm, with a range of 10 nm to 0.2 μm being further preferable.

In the case of the wet film-forming method, a thin film is formed by dissolving or dispersing materials forming each layer in an appropriate solvent such as ethanol, chloroform, tetrahydrofuran and dioxane. Any of the above-mentioned solvents can be used. Further, in any of the organic thin film layers, an appropriate resin or an appropriate additive may be used in order to improve film-forming properties and to prevent generation of pinholes in the film or for other purposes. Usable resins include insulative resins such as polystyrene, polycarbonate, polyarylate, polyester, polyamide, polyurethane, polysulfone, polymethyl methacrylate, polymethyl acrylate, cellulose or the like and copolymers thereof, photoconductive resins such as poly-N-vinylcarbazole and polysilane and conductive resins such as polythiophene and polypyrrole. As examples of the additive, antioxidants, UV absorbers, plasticizers or the like can be given.

The organic EL device of the invention can be suitably used as a planar emitting body such as a flat panel display of a wall-hanging television, backlight of a copier, a printer or a liquid crystal display, light sources for instruments, a display panel, a navigation light, or the like. The material of the invention can be use not only in an organic EL device but also in the field of an electrophotographic photoreceptor, a photoelectric converting element, a solar cell and an image sensor.

### EXAMPLES

The following compounds were synthesized and used in Examples and Comparative Examples.

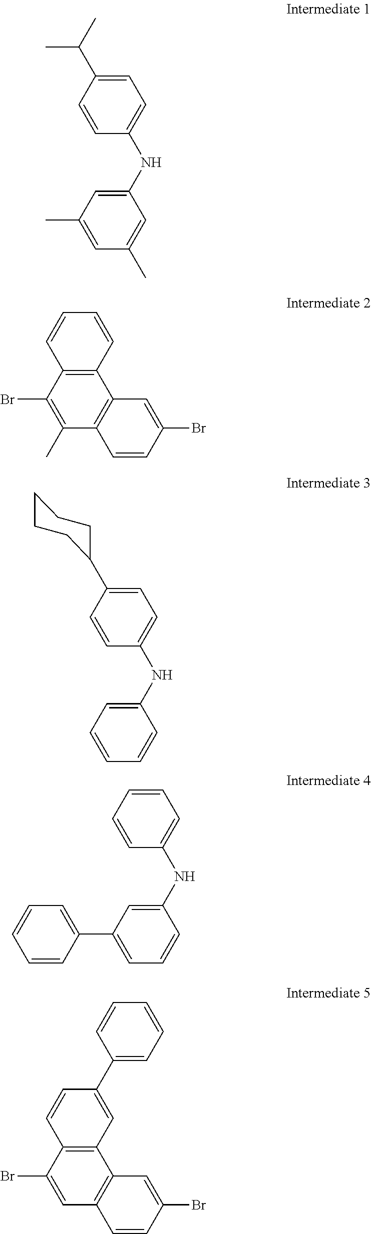

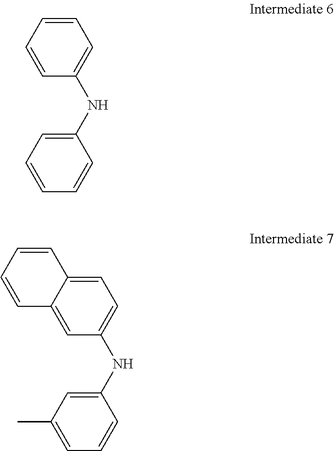

### Synthesis Example 1

### Synthesis of Compound (D-1)

Under an argon atmosphere, 5.0 g (14.9 mmol) of 3,9-dibromophenanthrene, 8.6 g (35.8 mmol) of Intermediate 1, 273 mg (0.298 mmol) of tris(dibenzylideneacetone)dipalladium, 241 mg (1.19 mmol) of tri-tert-butylphosphine, 4.3 g (44.7 mmol) of sodium(tert)butoxide and 40 mL of toluene were placed and stirred at 80° C. for 8 hours.

After cooling to room temperature, the mixture was filtered through cellite. The solution thus obtained was purified by short column chromatography (hexane/toluene). The resulting solids were recrystallized in toluene/ethanol and dried under reduced pressure to obtain 4.4 g of yellowish-white solids. As a result of FD-MS (field desorption mass spectrometry), the resulting solids were confirmed to be the compound D-1.

### Synthesis Example 2

### Synthesis of Compound (D-2)

Compound D-2 was synthesized in the same manner as in the synthesis of compound D-1, except that Intermediate 2 was used instead of 3,9-dibromophenanthrene and Intermediate 3 was used instead of Intermediate 1. As a result of FD-MS, the resulting solids were confirmed to be the compound D-2.

### Synthesis Example 3

### Synthesis of Compound (D-3)

Compound D-3 was synthesized in the same manner as in the synthesis of compound D-1, except that Intermediate 4 was used instead of Intermediate 1. As a result of FD-MS, the resulting solids were confirmed to be the compound D-3.

### Synthesis Example 4

### Synthesis of Compound (D-4)

Compound D-4 was synthesized in the same manner as in the synthesis of compound D-1, except that Intermediate 5 was used instead of 3,9-dibromophenanthrene and Intermediate 6 was used instead of Intermediate 1. As a result of FD-MS, the resulting solids were confirmed to be the compound D-4.

### Synthesis Example 5

### Synthesis of Compound (D-5)

Compound D-5 was synthesized in the same manner as in the synthesis of compound D-1, except that Intermediate 7 was used instead of Intermediate 1. As a result of FD-MS, the resulting solids were confirmed to be the compound D-5.

### Example 1

On a glass substrate of 25×75×1.1 mm, a 120 nm-thick transparent electrode formed of indium tin oxide was provided. After subjecting to UV-ozone cleaning, the glass substrate was mounted in a vacuum vapor deposition apparatus.

First, a 60 nm-thick film formed of N′,N″-bis[4-(diphenylamino)phenyl]-N′,N″-diphenylbiphenyl-4,4′-diamine was deposited as a hole-injecting layer. Then, a 20 nm-thick film formed of N,N,N′,N′-tetrakis(4-biphenyl)-4,4′-benzidine was deposited thereon.

Subsequently, 10,10′-bis[1,1′,4′,1″]terphenyl-2-yl-9,9′-bianthracenyl and the above-mentioned compound (D-1) were co-deposited in a weight ratio of 40:2 to form a 40 nm-thick emitting layer.

Next, as an electron-injecting layer, a 20 nm-thick film formed of tris(8-hydroxyquinolinato)aluminum was deposited. Then, a 1 nm-thick film formed of lithium fluoride was deposited, and a 150 nm-thick film formed of aluminum was deposited. The aluminum/lithium fluoride layer functions as a cathode. Thus, the organic EL device was fabricated.

The device obtained was subjected to current test. At a voltage of 6.4V and a current density of 10 mA/cm², blue emitting light (emission maximum wavelength: 457 nm) having a luminous efficiency of 6.4 cd/A and a luminance of 642 cd/m² was obtained.

### Examples 2 to 5

Organic EL devices were fabricated in the same manner as in Example 1, except that compounds shown in Table 1 were used instead of the compound (D-1). The results were shown in Table 1.

TABLE 1

| | | | | | | Emission | |
|---|---|---|---|---|---|---|---|
| | | Current | | Luminous | | maximum | Color |
| | | density | Voltage | efficiency | Luminance | wavelength | of emitted |
| | Compound | (mA/cm²) | (V) | (cd/A) | (cd/m²) | (nm) | light |
| Example 1 | D-1 | 10 | 6.4 | 6.4 | 642 | 457 | Blue |
| Example 2 | D-2 | 10 | 6.2 | 6.7 | 669 | 458 | Blue |
| Example 3 | D-3 | 10 | 6.6 | 6.3 | 633 | 455 | Blue |
| Example 4 | D-4 | 10 | 6.0 | 6.7 | 670 | 458 | Blue |
| Example 5 | D-5 | 10 | 6.6 | 6.3 | 631 | 456 | Blue |
| Com. Ex. 1 | H-1 | 10 | 6.2 | 3.1 | 311 | 451 | Blue |
| Com. Ex. 2 | H-2 | 10 | 6.3 | 2.8 | 283 | 454 | Blue |

### Comparative Examples 1 and 2

Organic EL devices were fabricated in the same manner as in Example 1, except that compounds shown in Table 1 were used instead of the compound (D-1). The results were shown in Table 1.

### INDUSTRIAL APPLICABILITY

The organic EL device using an aromatic amine derivative of the invention has a practically sufficient luminance at a low applied voltage and has a high luminous efficiency. Thus, it is useful as a light source such as a planar emitting body of a wall-hanging television and a backlight of display.

The contents of the above-described documents are herein incorporated by reference in its entirety.

1. An aromatic amine derivative represented by the following formula (1): 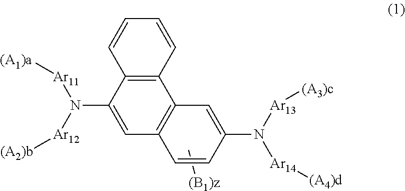
wherein Ar₁₁ to Ar₁₄ are independently a substituted or unsubstituted aryl group having 5 to 50 ring carbon atoms, or a substituted or unsubstituted heterocyclic group having 5 to 50 ring carbon atoms,
A₁ to A₄ are independently a hydrogen atom, a substituted or unsubstituted alkyl group having 1 to 50 carbon atoms, a substituted or unsubstituted aryl group having 5 to 50 ring carbon atoms, a substituted or unsubstituted aralkyl group having 6 to 50 ring carbon atoms, a substituted or unsubstituted cycloalkyl group having 3 to 50 ring carbon atoms, a substituted or unsubstituted alkoxyl group having 1 to 50 carbon atoms, a substituted or unsubstituted aryloxy group having 5 to 50 carbon atoms, a substituted or unsubstituted arylamino group having 5 to 50 ring carbon atoms, a substituted or unsubstituted alkylamino group having 1 to 20 carbon atoms, a substituted or unsubstituted heterocyclic group having 5 to 50 ring carbon atoms, a substituted or unsubstituted silyl group, a cyano group or a halogen atom,
B₁s are a hydrogen atom, a substituted or unsubstituted alkyl group having 1 to 50 carbon atoms, a substituted or unsubstituted aryl group having 5 to 50 ring carbon atoms, a substituted or unsubstituted aralkyl group having 6 to 50 ring carbon atoms or a substituted or unsubstituted cycloalkyl group having 3 to 50 ring carbon atoms,
a to d are independently an integer of 0 to 5 and when each of a to d is 2 or more, A₁s to A₄s may independently be the same or different and may bond to each other to form a saturated or unsaturated ring, and
z is an integer of 0 to 8 and when z is 2 or more, B₁s may independently be the same or different.

2. The aromatic amine derivative according to claim 1, wherein Ar₁₁ to Ar₁₄ in the formula (1) are independently a phenyl group or a naphthyl group.

3. The aromatic amine derivative according to claim 1, wherein A₁ to A₄ in the formula (1) are independently an alkyl group having 1 to 4 carbon atoms or an aryl group having 6 to 10 ring carbon atoms, and a to d are independently 1 or 2.

4. The aromatic amine derivative according to claim 1, wherein B₁s in the formula (1) are independently a hydrogen atom, an alkyl group having 1 to 4 carbon atoms or an aryl group having 6 to 10 ring carbon atoms, and z is 1 to 4.

5. The aromatic amine derivative according to claim 1, which is a doping material for an organic electroluminescence device.

6. An organic electroluminescence device comprising:
an anode, a cathode, and
one or plural organic thin film layers comprising at least an emitting layer between the anode and the cathode,
wherein at least one of the organic thin film layers comprises the aromatic amine derivative according to claim 1 singly or in the form of a mixture.

7. The organic electroluminescence device according to claim 6, wherein the emitting layer comprises the aromatic amine derivative singly or in the form of a mixture.

8. The organic electroluminescence device according to claim 6, wherein the emitting layer comprises the aromatic amine derivative in an amount of 0.1 to 20 weight %.

9. The organic electroluminescence device according to claim 6, which emits blue light.
